(19) United States
(12) Patent Application Publication
(10) Pub. No.: US 2008/0044892 A1
(43) Pub. Date: Feb. 21, 2008

(54) Air pollutants reduction system

(75) Inventor: Arthur C. Wu, Foster City, CA (US)

Correspondence Address: ARTHUR C. WU, 702 SOMERSET LANE, FOSTER CITY, CA, 94404, US

(73) Assignee: ARTHUR CHENG-HSIN WU, FOSTER CITY, CA (US)

(21) Appl. No.: 11/505,071

(22) Filed: Aug. 17, 2006

(51) Int. Cl. C12M 1/00

(52) U.S. Cl. 435/292.1

(57) ABSTRACT

Disclosed herein is a method for the air pollutants reduction system. The method comprises the reduction of air pollutants from their generated source, and biologically converting them into other forms of hydrocarbon materials and oxygen with the disclosed process.

### FIELD OF INVENTION

This invention related generally to processes and systems for use in the reduction of air pollutants from burning solid, liquid, and gas fuels, and more specifically related to a method and system for use in the reduction of air pollutants by the combined photosynthesis and ecological process in a disclosed system.

### BACKGROUND OF INVENTION

The present invention is directed to the emission reduction of exhaust from fuel burning, and in particular to biologically convert more than one component of air pollutants that include carbon dioxide, carbon monoxide, partially burned carbon based gases, nitrogen oxide including nitrogen monoxide and nitrogen dioxide, commonly referred to as NOₓ, sulfur oxide including sulfur monoxide and sulfur dioxide, commonly referred to as SOₓ, odors and air. As such, the present invention is directed to the air pollutants reduction, for example, electric power plant that burning fuels, municipal incinerator, Industrial or commercial fuel-burning facility, odors and gases generated from landfill, municipal downtown where emission pollutants and smog generated from motor vehicles, indoor theater, restaurant, hotel, and casino.

The addition to use carbon-based fuels as the fundamental energy source keeps generating huge amount of air pollutants into atmosphere. The amount of carbon dioxide keeps accumulating in the atmosphere and causes green house effect in the global scale. Mitigating global warming by removing carbon dioxides and other pollutants from atmosphere becomes a global concern and has been addressed intensively in the Kyoto Protocol.

Under normal condition, the earth surface shows a carbon dioxide concentration of about 0.033% or 330 ppm. It is estimated that a 1000-MW coal burning power plant can generate as high as 50 million barrels of carbon dioxide annually. Carbon sequestration becomes more and more important at the local point where it is generated in order to reduce the green house effects caused by high carbon dioxide concentration at local atmosphere environment.

In carbon-based fuel burning process, its chemical reaction generates carbon dioxide and may generate other unwanted products such as carbon monoxide, SOₓ, NOₓ, and other pollution materials. Photosynthesis is a way provided by nature to do carbon dioxide conversion biologically by plants that have chlorophyll. The amount of carbon dioxide absorbed and converted is limited per plant due to the conditions from its presented environment.

Several prior art patents have indicated mechanisms of carbon dioxide reduction. One of these, Wasserman U.S. Pat. No. 5,675,931, shows a plant tender and describes a control mechanism to supply water, light source and nutrients to indoors plant for the purpose of supporting for extended periods without any human intervention. The plant tender mainly can reduce carbon dioxide indoors with limited amount of capacity and is not capable to take care other polluting materials such as carbon monoxide, sulfur oxide, and nitrogen oxide.

Since light source is one of the fundamentals for photosynthesis, various prior art patents have also addressed the needs for light transmitting walls. One of these is shown in Clyde U.S. Pat. No. 4,446,236, that shows applying vessel structure as being made of transparent to light. However, the intensity of light did not considered for providing efficient photosynthesis.

Attempts have been made to take care air pollution that including carbon dioxide, sulfur dioxide and nitrogen dioxide. One of these, Kodo, et al. U.S. Pat. No. 6,083,740, shows a system for purifying a polluted air by using alga such as Spirulina to reduce carbon dioxide, nitrogen dioxide and sulfur dioxide in the polluted air and generates oxygen. When carbon dioxide, nitrogen dioxide, sulfur dioxide dissolve in water, they form carbonic acid, nitric acid, and sulfurous acid respectively. All these acid compounds can increase the water acidity level and it is contradict with common knowledge of maintaining the pH of water within alkaline range of 8.5 to 11 as disclosed by prior art. Furthermore, the prior art requires removing and filtering out Spirulina continuously when Spirulina reaching a predetermined size. This is due to large size of Spirulina can block light intensity while prior art at the same time requires increasing the Spirulina concentration in the cultured fluid.

In the industrial and commercial application, there is a need to have an air pollutants reduction system to take care various types of air pollutants from combustion exhaust. For this reason, there remains substantial room for improvement in the field.

### SUMMARY OF THE INVENTION

The present invention describes an air pollutants reduction system that forms an ecological process including photosynthesis and biological activities, and is capable of reducing various air pollutants from the exhaust of carbon-based fuels burning.

It is an object of the present invention to biologically converting air pollutants into nutrients, hydrocarbon materials, and oxygen to reduce the actual amount of air pollutants into atmosphere.

It is another object of the present invention to efficiently reducing carbon dioxide discharge from combustion exhaust for industrial application.

It is a further object of the present invention to provide an air pollutants reduction system that is energy efficient, trouble free to operate to reduce the cost of maintenance.

It is still another object of present invention to provide an air pollutants reduction system that has an economic incentive to produce commercial by-products for covering the operation cost.

These and other objects and advantages of the present invention will become clear to those skilled in the art upon review of the following specification, the accompanying drawings and the appended claims.

### BRIEF DESCRIPTION OF THE DRAWINGS

FIG. 1 is an overall diagrammatical view of an air pollutants reduction system according to the present invention.

### DETAILED DESCRIPTION OF THE PREFERRED EMBODIMENTS

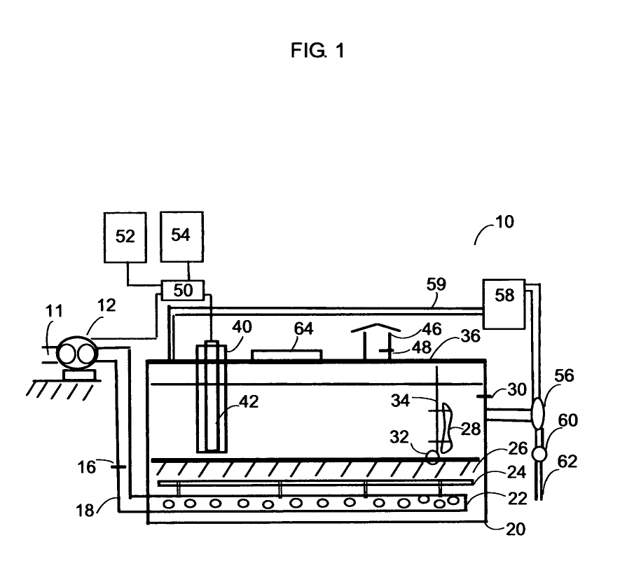

A preferred embodiment of the air pollutants reduction system is illustrated in a schematic diagram in FIG.1 and is referred to by the general reference character 10. In this illustration it may be seen that the air pollutants reduction system 10 is adapted to receive inflow of mixture of air pollutants 11 that containing the carbon monoxide, carbon dioxide, partially burned carbon gases and solid particles, NOₓ, SOₓ, odors and air, through blower 12.

Blower 12 delivers mixture of air pollutants 11 through air pipe 18 into tank 20. A sensor device 16 is used to analyze the compositions of mixture of air pollutants 11. Tank 20 can be in a cylindrical shape, a rectangular shape, or other shapes to fit into the operation requirements. Furthermore, tank 20 can be made of transparent material to allowing light passing through. A perforated pipe 22 connects with air pipe 18 to distribute mixture of air pollutants 11 inside the lower portion of tank 20. Perforated pipe 22 can be a straight pipe, a circular shape pipe, or a branch shape pipe with lots of fine holes on its surface.

Above the location of perforated pipe 22, a deflector 24 is positioned to reduce or block out foreign solid objects entering into perforated pipe 22. The lower portion of tank 20 is filled with mixture of soils 26 where comprising of stone, sand, soil and the likes. The mixture of soils 26 is preferred to be calcium and minerals rich. Minerals can include iron, magnesium, potassium, etc. For example, some beach sands have high calcium content in nature. The perforated pipe 22 and deflector 24 are submerged inside the mixtures of soils 26.

Above mixture of soils 26, a group of photosynthetic organisms 28 are planted. Photosynthetic organisms 28 can be algae or aquatic plants. Tank 20 is filled with water to a position indicator 30 that maintaining water level above the top portion of photosynthetic organisms 28. The water can be salt water if the photosynthetic organisms 28 belong to seawater type, or can be fresh water if the photosynthetic organisms 28 belong to freshwater type.

To accommodate buoyancy force in water especially alga has no root, an anchor device 32 is placed on the surface of mixture of soils 26 and is tied with a rope 34. The other end of rope 34 is tied to the tank cover 36. Along the rope 34, alga or photosynthetic plant is attached. By placing anchor device 32 and placing photosynthetic organisms 28 on rope 34, the positions of a group of photosynthetic organisms 28 can be arranged and densely packed in order to maximize the growing density of photosynthetic organisms 28 within the given space of tank 20. The photosynthetic organisms 28 on rope 34 can be checked manually by pulling up rope 34, and to be removed or replaced if needed.

A tube 40 made of transparent material is placed to enter into water and among photosynthetic organisms 28. Inside tube 40, a lighting device 42 that it either can be a light generating device such as incandescent light, illuminating light or a light transmitting device such as fiber optic, or can be a combined device of incandescent light and fiber optic, radiates light into tank 20. The quantity of tube 40 and lighting device 42 into tank 20 can be calculated and arranged to provide the needed light intensity for photosynthetic organisms 28 for the purpose of maintaining sufficient growth rate of photosynthetic organisms 28.

When the inflow of mixture of air pollutants 11 distributes through perforated pipe 22, it forms small air bubbles into tank 20. The location of deflector 24 forces air bubbles to be widely spread into mixture of soils 26. As described above mixture of air pollutants 11 contain more than one type of polluted materials such as carbon dioxide, carbon monoxide, NOₓ, SOₓ, partially burned carbon-based gases or solid particles, odors, air, etc. The mixture of soils 26 perform as several layers of fine mesh screens to break down the air bubbles into smaller sizes. This can improve dissolving rates of mixture of air pollutants 11 into water.

For the carbon dioxide portion, it dissolves into water and forms carbonic acid. The solubility of carbon dioxide is temperature and pressure dependent. In general, at room temperature solubility can reach as high as 90 cubic centimeter of carbon dioxide per 100 ml of water. For the SOₓ portion, sulfur monoxide turns into sulfur dioxide when reacting with oxygen. Sulfur dioxide dissolves into water and forms sulfurous acid. For the NOₓ portion, nitrogen monoxide quickly turns into nitrogen dioxide when reacting with oxygen. Nitrogen dioxide dissolves in water and forms nitric acid. All the dissolved particles mentioned above such as carbonic acid, sulfurous acid and nitric acid have the tendency to attach on particle surfaces of mixture of soils 26. This molecular attraction due to large molecular weight of solid particle surface of mixture of soils 26 and the chemical attraction due to the particles of mixture of soils 26 are calcium and minerals rich.

For the carbon monoxide portion, carbon monoxide has relatively low solubility in water, and some of them can be attached to the particle surfaces of the mixture of soils 26 and be reacted with oxygen to form carbon dioxide. Other portions of air pollutants such as partially burned carbon-based gases, solid particles and odors may or may not dissolve into water depending on their particular molecular structures.

The layer of mixture of soils 26 becomes a buffer zone to store more than one components of mixture of air pollutants 11 and it even may attach oxygen and nitrogen. Portion of mixture of air pollutants 11 enters into upper portion of tank 20 directly during the inflow feeding process described above. However, the layer of mixture of soils 26 functions as a buffer zone to attach and store components of mixture of air pollutants 11 and gradually releases and diffuses them into water. Additionally the dissolved components of mixture of air pollutants 11 react with the particles of mixture of soils 26 and release calcium and mineral compound to mitigate the acidity of water in tank 20.

Inside tank 20, it forms an ecological system. Photosynthetic organisms 28 carry out photosynthesis by utilizing the light radiated from the lighting device 42 in the presence of dissolved carbon dioxide, sulfur dioxide and nitrogen dioxide, released calcium and minerals compounds from mixture of soil 26 as nutrients to grow, to become part of body structure, and to generate oxygen.

Another preferred embodiment of the present invention is to add soil-borne microorganisms into the mixture of soils 26. The mixture of soils 26 is a good breeding ground for soil-borne microorganisms. Soil-borne microorganisms including aerobic type and facultative type can be inoculated either from local indigenous or cultured. The soil-borne microorganisms can be the combination of a wide variety of microorganisms from nature or from commercially available products. Soil-borne microorganisms can include Bacillus, Pseudomonas, Flavobacterium, Streptomyces, cyanobacteria, etc. The selection of soil-borne microorganisms is based on beneficial and also can work together with photosynthetic organisms 28, and performs as an overall function with respect to the system 10. The population of soil-borne microorganisms in mixture of soils 26 is relatively high, and diffuses into water and stays on the surfaces of photosynthetic organisms 28.

The soil-borne microorganisms in the mixture of soils 26 consume any available nutrient that including chemical compounds such as sulfur dioxide and nitrogen dioxide attached on the particle surfaces of mixture of soils 26; calcium and mineral particles such as magnesium, copper, iron either originally from mixture of air pollutants 11 or from mixture of soils 26; plus air and water. The soil-borne microorganisms extract energy from them and to form microbial by-products.

The microbial by-products mentioned above plus dissolved carbon dioxide, sulfur dioxide, and nitrogen dioxide in water become main food sources for photosynthetic organisms 28. With the available light radiated from lighting device 42, photosynthesis is taking place to let photosynthetic organisms 28 take in energy, to grow, to consume components of mixture of air pollutants 11 and to generate oxygen.

One of advantages of adding soil-borne microorganisms into tank 20 is to mitigate the water acidity level. Soil-borne microorganisms have tendency to adjust their living environment to their natural living conditions in which the pH ranges from 5.5 to 6.5. During the process of inflow of mixture of air pollutants 11 into tank 20, the water acidity level usually increases. Soil-borne microorganisms are forced to extract more calcium and minerals from mixture of soils 26 for the purpose of mitigating water acidity level.

The mixture of soils 26 plus soil-borne microorganisms show a function of control release mechanism. For example, the sulfur contents in mixture of air pollutants 11 increase temperately, and water acidity level correspondingly increases. More alkaline compounds are released from mixture of soils 26 to adjust water pH level by the activities of soil-borne microorganisms. Additionally the soil-borne microorganisms take in more sulfur compounds into their cells as food source. When water acidity level back to their preferred living range for soil-borne microorganisms, the amount of alkaline compounds released from mixture of soils 26 reduces. At this moment, the soil-borne microorganisms release more sulfur compounds that previously stored in their cells.

At some conditions, the pH of water in tank 20 may be distorted toward alkalinity. For example, the inflow of mixture of air pollutants contains large amount of calcium, or magnesium. Soil-borne microorganisms instinctively produce more acid compounds to adjust water pH level to their preferred range.

This behavior of adjusting water pH level for the purpose of reaching preferred living environment in tank 20 can create a better living environment for soil-borne microorganisms as well as for photosynthetic organisms 28. This control release mechanism can avoid the decay of photosynthetic organisms 28 that have limited tolerance up to a certain level of acidity. The mixture of soils 26 can be replaced when the contents of calcium and mineral are depleted.

In summary, with lighting device 42 radiating light into tank 20, photosynthesis from photosynthetic organisms 28 and biological activities from soil-borne microorganisms in the mixture of soils 26 form a complex ecological system. The mixture of air pollutants 11 entering into tank 20 becomes food sources either for soil-borne microorganisms or photosynthetic organisms 28, or both. The consumed mixture of air pollutants 11 materials through this ecological arrangement become part of cell structure of photosynthetic organisms 28, part of nutrient released from photosynthetic organisms 28, part of cell structure of soil-borne microorganisms, and microbial metabolism by-products and nutrients generated from soil-borne microorganisms.

The water and mixture of soils 26 in tank 20 function as an absorbing mechanism to store and release mixture of air pollutants 11 that attached on mixture of soils 26. The anchor device 32 and rope 34 function as a positioning device for placing and arranging photosynthetic organisms 28. The soil-borne microorganisms in mixture of soils 26 function as a water pH adjusting mechanism toward their natural desired living environment. Photosynthetic organisms 28 and soil-borne microorganisms consume parts of mixture of air pollutants 11 as nutrients. With the presence of lighting device 42 radiating sufficient light into water, photosynthetic organisms 28 perform photosynthesis by taking in carbon dioxide, other parts of mixture of air pollutants, nutrients released from mixture of soils 26, and microbial by-products from soil-borne microorganisms and generate amino acid, glucose and oxygen.

In the present system 10, when the supplied nutrients from soil-borne microorganisms, from mixture of air pollutants 11, and from mixture of soils 26, become sufficient to support photosynthetic organisms 28, the present system 10 reaches an ecological balance, and become self-sustainable for long term operation.

As indicated in present system 10, the extra air and water moisture on the top portion of tank 20 release through exhaust pipe 46. A sensor-reading device 48 records the composition of air in the exhaust pipe 46, and compares the reading with respect to a sensor device 16 at the inflow. The concentration of mixture of air pollutants 11 is relatively high around the bottom portion of tank 20, and gradually reduces its concentration through the level of tank 20.

In present system 10 although tank 20 and tank cover 31 can be made of transparent materials to let outside light passing through, for the purpose of maximizing photosynthesis for photosynthetic organisms 28, it is preferred to install multi-sets of tube 40 with lighting device 42 as to increase light intensity in water.

In general, sunlight intensity is within range of 2000 to 4000 lux. During sunrise and sunset period, sunlight intensity is low and around noontime sunlight intensity reaches its peak. To assure the photosynthesis process actively going in tank 20, the lighting device 42 inside tube 40 is suggested to maintain light intensity above 4000 lux. With sufficient light available and other food sources supplied from mixture of soils 26 and water, photosynthetic organisms 28 can have a high growth rate and can contribute more consumption of dissolved mixture of air pollutants 11 in water. If there is a need, particularly in the setup period, extra food source such as urea can add into tank 20.

As illustrated in present system 10, a battery device 50 connects to a lighting device 42 and blower 12 to supply electric power. Furthermore, battery device 50 connects to a set of solar panel 52 in which solar energy is converted into electric energy. Additionally the battery device 50 is connected to a power source 54, so when solar electric power cannot fulfill needs, the power source 54 supplies the needed portion.

A water pump 56 is used to circulate water in tank 20 and is used to feed the water trough a filter 58, and to circulate back through a circulation pipe 59. This filtering action removes unwanted small objects such as part of body structure of photosynthetic organisms 28, solid particles, etc. to reduce turbidity in tank 20.

Additionally, this action can generate circulating motion in water and can provide more mixing and longer resident time for mixture of air pollutants 11 in water. The water circulation motion makes water and contained mixture of air pollutants 11 flow directly toward the photosynthetic organisms 28 that are placed on the rope 34. This water circulation motion makes photosynthetic organisms 28 take in more nutrients. When the water level inside tank 20 below the mark of position indicator 30, the valve 60 of water pipe 62 is open to supply makeup water. The supplied water also is used to adjust the bulk temperature in tank 20.

A manhole 64 is placed on the top of tank 20 for system inspection and to harvest photosynthetic organisms 28 inside tank 20 when photosynthetic organisms 28 are grown to a certain stage.

As will be understood by those skilled in the art, various modifications and alterations of the specific structure described above as constituting the preferred embodiment may be utilized with acceptable results. For example, the specific structures of the pipes, chutes, valves, and tubes described above may be modified substantially while still retaining the primary functional characteristics and providing results which are improved over those of the prior art. Certainly, the dimensions and materials may also be modified, particularly depending upon the specific purpose for which the air pollutants reduction system 10 is intended to be used.

One component of the system 10 which is significant is the selection of photosynthetic organisms 28. As described above, photosynthetic organisms 28 can be algae or aquatic plants.

Spirulina is a type of blue-green algae found in most lakes and ponds. Spirulina is considered a complete protein because well over half of it consists of amino acids—the building blocks of protein. From per unit weight point of view, Spirulina shows remarkable capacity to take in carbon dioxide and to generate oxygen during photosynthesis. However, Spirulina has a tendency to floating on water surface due to its specific gravity. Therefore, it is very difficult to let Spirulina be uniformly distributed in a closed container unless keeping in a continuous stirring motion that may or may not be feasible in a practical application.

In an industrial case, the total air pollutants reduction amount is one of major requirements from engineering point of view. A container with a certain set of dimensions is designed to reach a predetermined amount of air pollutants reduction. If the height of the container is not high enough whether it is vertical or inclined, the resident time for air bubbles is too short to have a meaningful photosynthesis. When Spirulina alone is placed in a container, Spirulina densely floats on water surface in the top portion of container and has no any presence in the middle and bottom portion of container. The limitation of providing sufficient light radiating on top portion of container makes the total amount of air pollutants reduction fail to achieve its predetermined requirement.

For this industrial case, seaweed may be one of the better choices. Seaweed is a type of marine algae. Seaweeds are found on both sandy and rocky beaches. Many can tolerate low salinity and can colonize area where rivers meet the sea. Seaweeds can be affixed on rope 34, and can be densely arranged through multi-sets of rope 34 in tank 20 while allowing multi-sets of lighting device 42 radiating light widely in tank 20.

From per unit weight consideration, Spirulina has a higher value compared with seaweed to take in air pollutants and to generate oxygen. Form total air pollutants reduction consideration, seaweed can reach the total amount of air pollutants reduction predetermined by engineering requirements.

For the industrial case mentioned above, combination of Spirulina and seaweed is another choice, that is, seaweed is tied on the rope 34 and let Spirulina floats on the upper portion of water in tank 20.

One of the advantages of the present invention 10 is the efficiency factor. Tightly planting photosynthetic organisms 28 in a confined space such as tank 20, with calculated supply of light radiation from lighting device 42, the photosynthesis can reach its biological maximum performance level for a given type of photosynthetic organisms 28 and more than one type of mixture of air pollutants 11 can be consumed for the purpose of reaching air pollutants reduction. The engineering scale up for a practical application becomes more straightforward.

Another advantage of the present invention 10 is the economic factor. When photosynthetic organisms 28 grow and reach a certain stage, those extra amounts of photosynthetic organisms 28 cannot contribute the extra benefit of air pollutants reduction and are collected from tank 20. If the scale of operation is large enough, that is, multi-sets of tank 20 are placed in one location, the total collection of photosynthetic organisms 28 can be processed further to make animal feed or organic food. The sales income from harvested photosynthetic organisms 28 can cover the daily expense and also may amortize the original investment.

Those skilled in the art will readily recognize that numerous other modifications and alterations of the specific structures, dimensions, materials and components may be made without departing from the spirit and scope of the invention. Accordingly, the above disclosure is not to be considered as limiting and the appended claims are to be interpreted as encompassing the entire scope of the invention.

### INDUSTRIAL APPLICABILITY

The air pollutants reduction system 10 according to the present invention is adapted to be utilized in a wide variety of industrial and field applications wherein it is desired to covert mixture of air pollutants 11 into other type of hydrocarbon materials and oxygen. The system 10 is adapted particularly for combustion exhaust to remove and reduce air pollutants.

The following operation test illustrates the advantages of air pollutants reduction system 10. Heavy oil was used as fuel for a furnace and its exhaust contained smoke and mixture of air pollutants 11. The exhaust flew into a web scrubber to remove soot and solid particles, and its temperature was reduced to about 28 degrees Celsius (about 82.4 degrees Fahrenheit). Then the initial treated exhaust mixed with fresh air were driven by blower 12 and pushed into air pipe 18. At this stage, the mixed air after blower 12 reached to about 32 degrees Celsius (about 89.6 degrees Fahrenheit). The position of blower 12 was placed higher than the water level indicator 30 to avoid backflow water.

Large amount of air bubbles were formed on bottom surface area of tank 20. Air bubbles initially went straight up when leaving the surface of mixture of soils 26 and turned into a circulating motion caused by water pump 56.

The mixture of soils 26 has inoculated by soil-borne microorganisms through a commercial available product. For example, the agriculture microbial product supplied from VITABIO, Inc. was chosen for this application.

The selection of photosynthetic organisms 28 for this field test was green seaweed. The water in tank 20 was maintained at low salinity level, about 50% of salinity compared with seawater. The pH in tank 20 was maintained around 6.0. Several seaweeds were secured on one rope 34, and a metal made hook object was used as anchor device 32. The distance between ropes was about 7 centimeters (about 3 inches). Multi-sets of tube 40 and lighting device 42 were arranged in tank 20. Each lighting device 42 provided over 4000 lux of light intensity. The water and air bubbles mixed together and maintained in a circulating motion. This circulating motion caused air bubbles flowing toward seaweeds.

After the operation reaching steady state, the readings from sensor device 16 showing carbon dioxide concentration is 1.80%, and carbon dioxide concentration at exhaust pipe 46 is 1.08%. This is 40% of reduction in carbon dioxide, and the readings comparisons show carbon monoxide and NO₂ reduction in the range of 14% and 26%. The SO₂ concentration at inlet location of sensor device 16 is very low, less than 0.05 ppm. The SO₂ concentration at exhaust pip 46 is zero.

In light of the many efficiencies and advantages of the air pollutants reduction system 10 of the present invention, it is expected to appeal to a great number of potential users. Efficiency of operation, and minimum down time for cleaning, substantial automated, provides advantages that make the air pollutants reduction operation efficient. Accordingly, it is expected that the air pollutants reduction system 10 according to the present invention will have industrial applicability and environmental utility which are both wide spread and long lasting.

1. A system for reducing polluted air, comprising:
a tank containing water, mixture of soils and photosynthetic organism;
an arranging means including a rope for positioning said photosynthetic organism along said rope;
a radiation means for providing light with intensity over 4000 lux into said water;
an feeding means for supplying said polluted air into said mixture of soils for breaking down sizes of said polluted air, and dissolving components of said polluted air into acid compounds;
a mixing means for circulating said water, said acid compounds flowing toward said photosynthetic organism;
wherein said photosynthetic organism consumes said polluted air and said acid compounds and produces oxygen; and
wherein said mixture of soils contain calcium and minerals; and
wherein said mixture of soils release said calcium and minerals for reducing acidity of said water.

2. The system of air pollutants reduction of claim 1, wherein said photosynthetic organism is an alga.

3. The system of air pollutants reduction of claim 1, wherein said photosynthetic organism is an aquatic plant.

4. The system of air pollutants reduction of claim 2, wherein said alga is seaweed.

5. An air purification system, comprising:
a tank containing water, mixture of soils, photosynthetic organism and soil-borne microorganisms;
an air supply means for delivering polluted air into said mixture of soils;
a position means for placing said photosynthetic organism;
a lighting means for radiating light to said photosynthetic organism;
a circulation means for circulating said water;
wherein said mixture of soils provide housing for soil-borne microorganisms; and
wherein said polluted air is purified by said photosynthetic organism and said soil-borne microorganisms; and
wherein said soil-borne microorganisms and said mixture of soils adjust pH of said water toward a natural living range of said soil-borne microorganisms.

6. The air purification system of claim 5 wherein said photosynthetic organism is an alga.

7. The air purification system of claim 5 wherein said photosynthetic organism is an aquatic plant.

8. The air purification system of claim 5 wherein said lighting means has light intensity over 4000 lux.

9. The air purification system of claim 6 wherein said alga is seaweed.

10. A system for generating purified air through photosynthesis and biological processes, comprising:
a tank containing water, mixture of soils, photosynthetic organism, and soil-borne microorganisms;
an anchor means including a rope for placing said photosynthetic organism for maximizing quantity of said photosynthetic organism inside said tank;
a light source means including a tube made of transparent material and a lighting device inserted into said tube for providing light with intensity over 4000 lux into said water;
an infeed means including a perforated pipe for feeding in polluted air into said tank;
a circulation means including a pump and a filter;
wherein said pump mixes said polluted air and said water for generating circulating motion inside said tank; and
wherein said filter removes solid materials from said water; and
wherein said soil-borne microorganisms utilize said polluted air and calcium and minerals from said mixture of soils as food sources for generating microbial by-products and for adjusting the pH of said water; and
wherein said photosynthetic organism performs photosynthesis with consuming said polluted air and said microbial by-products for generating said purified air.

11. The system of claim 10, wherein said photosynthetic organism is an alga.

12. The system of claim 10, where said photosynthetic organism is an aquatic plant.

13. The system of claim 11, wherein said alga is seaweed.
